(19) United States
(12) Patent Application Publication
(10) Pub. No.: US 2005/0203366 A1
(43) Pub. Date: Sep. 15, 2005

(54) Neurological event monitoring and therapy systems and related methods

(76) Inventors: John P. Donoghue, Providence, RI (US); Mijail D. Serruya, Providence, RI (US); J. Christopher Flaherty, Topsfield, MA (US); Brian W. Hatt, Salt Lake City, UT (US); Jon P. Joseph, Madison, WI (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/798,919

(22) Filed: Mar. 12, 2004

(51) Int. Cl. A61B 5/04

(52) U.S. Cl. 600/378; 607/46

(57) ABSTRACT

Systems and methods for detecting, monitoring, and/or treating neurological events based on, for example, electrical signals generated from the patient's body are disclosed. Various embodiments of the invention include a system for predicting occurrence of a neurological event in a patient's body. The system may include an implant configured to be placed in the body and detect signals indicative of an activity that precedes the neurological event, and a processing unit configured to process the detected signals so as to predict the neurological event prior to the occurrence.

### FIELD OF THE INVENTION

The invention relates to systems and methods for detecting, monitoring, and/or treating neurological events. In a particular embodiment, the invention relates to systems and methods for predicting a neurological event based on, for example, electrical signals generated from the patient's body and/or generating a signal used to treat the neurological event.

### DESCRIPTION OF THE RELATED ART

Recent advances in neurophysiology have allowed researchers to detect and study the electrical activity of highly localized groups of neurons located in a specific portion of the body with high temporal accuracy. The information in the sensed electrical activity may include a variety of information, including physiologic information and motor mapping information. These advances have created the possibility of extracting and processing that information and creating brain-machine interfaces (BMIs) that, for example, may allow treatment of certain neurological disorders.

For example, epilepsy is a common neurological disorder, and such brain-computer interfaces may be used to detect and treat epileptic symptoms. Epilepsy may be characterized as electro-physiologic abnormalities causing sudden recurring seizures or motor, sensory, or psychic malfunctioning. While the majority of epileptic patients may be effectively treated with anti-epileptic drugs (AED), many patients continue to have symptoms or side effects that seriously impair their quality of life, and may have to rely on a surgical solution to reduce or eliminate their symptoms.

While various surgical methods are currently available to treat the epileptic patients (e.g., resection of brain tissue to remove epileptic focus or stimulating the Vagus nerve to suppress a seizure), the single most valuable information to an epileptic patient may be predictive information indicating, for example, when a seizure might occur and with what probability. Such prediction capability may provide an epileptic patient with an opportunity to take appropriate responsive actions to suppress the forthcoming seizure or simply to avoid potentially dangerous situations by, for example, lying down on a bed, pulling a car over to the side of a road, or getting out of a shower. The predictive information of the epileptic seizure may also provide useful information to a physician to enable development of enhanced therapeutic methods, such as biofeedback, drug delivery, and stimulation, to suppress or delay the seizure or dampen its severity.

The seizure prediction, however, requires detailed understanding of how individual cells behave as a population prior to the actual occurrence of the seizure. For instance, seizures are often believed to be a population phenomenon in which a seizure focus fires an initiation signal that synchronizes activity in the rest of the brain, thereby blocking its normal function. Therefore, in addition to the knowledge of precise localization of the epileptic focus, each individual cell activity may have to be detected to predict seizure occurrence.

Various sensors have been used to detect electrical activity in a brain to identify the epileptic zone or focus. For example, noninvasive sensors, such as multi-channel electroencephalogram (EEG) sensors placed on the surface of a patient's scalp, have been used as simple BMI interfaces. EEG sensors, however, may not offer sufficient temporal or spatial resolution needed to fine grain the seizure focus or to detect single cell activity. Instead, EEG sensors detect mass fluctuations of averaged neuron activity and, therefore, provide much simpler, reduced forms of neuron activity information without providing information about the activity of single cells or their interactions.

Therefore, there is a need for advanced BMIs that may provide sufficient temporal or spatial resolution sufficient to accurately identify the location of a seizure focus and/or the temporal evolution of the shift from normal to seizure-like activity. This spatial and temporal resolution may require the ability to monitor individual neuron activity, so as to detect and characterize various seizure-inducing conditions (e.g., specific firing patterns of the neuron spikes) that can be used to predict seizure occurrences. Moreover, development of suitable algorithms or methods for use in connection with such advanced BMIs may be desirable to enhance the prediction capability of the BMIs and/or treatment of the epileptic symptoms.

### SUMMARY OF THE INVENTION

Therefore, an embodiment of the invention relates to a system and method that may predict a neurological event prior to its occurrence and generate various control signals that can be used to suppress or control the neurological event.

To attain the advantages and in accordance with the purpose of the invention, as embodied and broadly described herein, one aspect of the invention may provide a system for predicting occurrence of a neurological event in a patient's body. The system may comprise an implant configured to be placed in the body and detect signals indicative of an activity that precedes the neurological event, and a processing unit configured to process the detected signals so as to predict the neurological event prior to its occurrence.

In accordance with another aspect of the invention, the implant may be configured to be placed in a patient's brain. The implant may include at least one multi-electrode array, and the multi-electrode array may include a plurality of electrodes. The plurality of electrodes may be configured to penetrate into neural tissue of the brain to detect electrical signals generated from the neurons. The multi-electrode array may include at least one of a recording electrode, a stimulating electrode, and an electrode having recording and stimulating capabilities. The at least one multi-electrode array may be configured to detect electrical signals indicative of a neural activity preceding the neurological event.

In still another aspect of the invention, the implant may be configured to detect electrical signals generated from the neurons located proximate the implant. The processing unit may be configured to convert the detected electrical signals into a recognizable pattern. The recognizable pattern may include a formula describing a behavior of the neurons in time and space. The implant may also be configured to isolate individual neuron signals from neighboring neuron signals.

In yet still another aspect of the invention, the detected electrical signals generated from the neurons may include electrical spikes. The processing unit may be configured to characterize a pattern of the electrical spikes that represent a neural activity preceding the neurological event, so as to predict the occurrence of the neurological event.

According to another aspect of the invention, the implant may be configured to be placed proximate a neural focus in the brain that initiates the neurological event. The implant may be configured to detect local field potentials of the brain. Alternatively or additionally, the implant may be configured to detect electrocorticogram (ECOG) signals, electroencephalogram (EEG) signals, DC potentials, light, and/or acoustic waves generated from the neurons located proximate the implant.

In still another aspect of the invention, the implant may comprise a subdural grid having a plurality of electrode contacts and configured to be placed on a surface of the brain. The implant may also include at least one multi-electrode array. In another aspect, the implant may include a movement sensor configured to detect movement of the brain, a pressure monitoring device for monitoring pressure in the brain, a temperature monitoring device for monitoring temperature in the brain, and/or a magnetic resonance monitoring device for monitoring magnetic resonance intensity in the brain.

In accordance with another aspect of the invention, the processing unit may be configured to characterize the signals that represent the activity preceding the neurological event.

Another aspect of the invention may also provide a storage device for storing the signals that represent the activity preceding the neurological event. The processing unit may be configured to compare the detected signals with the signals stored in the storage device. The processing unit may include a recording device for recording the detected signals.

In still another aspect of the invention, the implant may be configured to detect biological or physiological signals generated within the patient's body. The system may also comprise a sensor for detecting other signals generated from the body, and the sensor may be configured to communicate with the processing unit. The processing unit may be configured to compare the signals detected by the implant and the other signals detected by the sensor.

In yet still another aspect of the invention, the processing unit may be configured to differentiate the signals indicative ot the activity that precedes the neurological event from signals resulting from normal activities.

Another aspect of the invention may provide a processing unit that may be configured to output information relating to a patient's condition with respect to the neurological event. The processing unit may include an indicator for conveying the information to the patient.

In still another aspect, an external device in communication with the processing unit may be provided. The external device may be configured to display the information relating to the patient's condition with respect to the neurological event. The processing unit may also be configured to receive an input signal from the external device. The external device may include at least one of a visual indicator and an auditory indicator.

In another aspect of the invention, the information may include a warning signal that the neurological event is expected to occur. Alternatively or additionally, the information may include a time remaining until the occurrence of the neurological event, an occurrence probability of the neurological event, and/or severity of the neurological event. In another aspect, the information may include a patient's current condition in comparison with a normal target condition. In still another aspect, the information may include instructions for preventing the neurological event from occurring.

According to another aspect of the invention, the information may include a stimulating signal provided to the patient to cause a movement of a portion of the patient's body. The stimulating signal is sent to the implant. In an aspect, the portion of the patient's body may be a finger.

In still another aspect of the invention, upon predicting the occurrence of the neurological event, the processing unit may be configured to generate a control signal to suppress, dampen, or delay the neurological event. The control signal may include an electrical current sent to a patient's brain to stimulate at least a portion of the brain. Alternatively or additionally, the control signal may be configured to stimulate a central nervous system and/or a peripheral nervous system. In another aspect, the system may include a drug delivery system. The processing unit may send a signal to the drug delivery system to deliver a therapeutic agent or drug to at least a portion of the patient's body.

In yet still another aspect of the invention, the processing unit, upon predicting the occurrence of the neurological event, may be configured to hyperpolarize at least a portion of the brain. In another aspect, the processing unit may send a DC bias current to a patient's brain to hyperpolarize the at least a portion of the brain.

In another aspect of the invention, the implant may include one or more electrodes and, upon predicting the occurrence of the neurological event, the processing unit may be configured to short the one or more electrodes.

In still another aspect of the invention, the system may provide a storage device containing a target signal indicative of the activity that precedes the neurological event. The target signal may include a database containing a set of previously detected signals indicative of the activity that precedes the neurological event. The processing unit may be configured to compare the detected signals with the target signal. The processing unit may be configured to modify the target signal.

In another aspect of the invention, the neurological event may be an epileptic symptom. The implant may be placed proximate an epileptic focus of the brain.

In still another aspect of the invention, the neurological event may be an undesired activity. The undesired activity may include a criminal activity. The implant may be configured to be placed in a brain and measure readiness potential of the brain, indicative of occurrence of the undesired activity.

Another aspect of the invention may provide a method for treating a neurological event in a patient. The method may include placing an implant in the patient's body, detecting signals indicative of an activity that precedes the neurological event, and predicting occurrence of the neurological event based on the detected signals.

In another aspect of the invention, the method may also include placing the implant in the patient's brain. The remote implant may include at least one multi-electrode array, and the multi-electrode array may include a plurality of electrodes. The plurality of electrodes may be configured to penetrate into neural tissue of the brain to detect electrical signals generated from the neurons. The multi-electrode array may include at least one of a recording electrode, a stimulating electrode, and an electrode having recording and stimulating capabilities. The method may also include detecting electrical signals with the multi-electrode array, where the electrical signals may be indicative of a neural activity preceding the neurological event.

Still another aspect of the invention may include detecting electrical signals generated from the neurons located proximate the implant. The method may also include processing the detected electrical signals to convert the signals into a recognizable pattern. The recognizable pattern may include a formula describing a behavior of the neurons in time and space. In another aspect of the invention, the method may include processing the detected electrical signals to isolate individual neuron signals from neighboring neuron signals. The detected electrical signals generated from the neurons may include electrical spikes.

In another aspect of the invention, the implant may be placed proximate a neural focus in the brain that initiates the neurological event. The implant is configured to detect local field potentials of the brain, electrocorticogram (ECOG) signals, electroencephalogram (EEG) signals, DC potentials, light, and/or acoustic waves generated from the brain.

In still another aspect of the invention, the implant may include a subdural grid having a plurality of electrode contacts, where the subdural grid may be placed on a surface of the brain. The implant may further include at least one multi-electrode array.

In yet still another aspect of the invention, the implant may include a movement sensor configured to detect absolute or relative movement of the brain, a pressure monitoring device for monitoring pressure in the brain, a temperature monitoring device for monitoring temperature in the brain, and/or a magnetic resonance monitoring device for monitoring magnetic resonance intensity in the brain. The method may include a step of processing the detected signals to characterize the signals that represent the activity preceding the neurological event.

Another aspect of the invention may provide a step of storing the signals that represent the activity preceding the neurological event into a storage device. The method may also include comparing the detected signals with the signals stored in the storage device. In still another aspect, the method may include comparing the detected signals with other signals detected by a sensor in the patient's body. In still another aspect of the invention, the method may include recording the detected signals. The detected signals may include biological or physiological signals generated within the patient's body. In yet still another aspect of the invention, the method may also include differentiating the signals indicative of the activity that precedes the neurological event from signals resulting from normal activities.

Another aspect of the invention may include outputting information relating to the patient's condition with respect to the neurological event. Outputting information may include conveying the information to the patient. The information may include a warning signal that the neurological event is expected to occur. Outputting information may also include communicating with an external device to convey the information. The external device may include at least one of a visual indicator and an auditory indicator. The information may include a time remaining until the occurrence of the neurological event, an occurrence probability of the neurological event, and/or severity of the neurological event. The information may include a patient's current condition in comparison with a normal target condition and/or instructions for preventing the neurological event from occurring.

In another aspect of the invention, outputting the information may include causing a movement of a portion of the patient's body. The step of causing the movement may include sending a stimulating signal to the implant. The portion of the patient's body may include a finger.

In still another aspect of the invention, the method may include generating, upon predicting the occurrence of the neurological event, a control signal for treating the patient. The control signal may control, suppress, dampen, and/or delay the neurological event.

In an aspect of the invention, generating a control signal may include generating a stimulating electrical current and sending the current to a portion of the patient's body. The portion of the patient's body may include the patient's brain. In another aspect of the invention, generating a control signal may include generating a signal to deliver a drug or a therapeutic agent to at least a portion of the patient's body.

In still another aspect of the invention, the method may include hyperpolarizing, upon predicting the occurrence of the neurological event, at least a portion of the patient's brain. Hyperpolarizing may include sending a DC bias current to the patient's brain to hyperpolarize the at least a portion of the brain.

In another aspect of the invention, the implant may include at least one electrode and, upon predicting the occurrence of the neurological event, the processing unit may be configured to short the at least one electrode.

Still another aspect of the invention may provide a target signal indicative of the activity that precedes the neurological event. The target signal may include a database containing a set of previously detected signals indicative of the activity that precedes the neurological event. The method may include comparing the detected signals with the target signal. In another aspect, the method may include modifying the target signal.

In another aspect of the invention, modifying the target signal may include performing an adaptive processing of the target signal. In an aspect, the adaptive processing may include determining whether the neurological event occurred, regardless of whether the occurrence was predicted, determining whether the occurrence or nonoccurrence of the neurological event was mistakenly predicted, and modifying the target signal based on whether the occurrence or nonoccurrence of the neurological event was mistakenly predicted.

In still another aspect of the invention, the neurological event may be an epileptic symptom. The method then may include placing the implant proximate an epileptic focus of a brain.

In another aspect of the invention, the method may include preprocessing the detected signal. Preprocessing may include measuring background signals and calibrating the detected signal based on the measured background signals. Alternatively or additionally, preprocessing may include at least one of noise filtering, impedance matching, rectifying, integrating, differentiating, discretizing, and amplifying the detected signals.

Still another aspect of the invention may provide a system for detecting a neurological event in a patient's body. The system may provide at least one electrode placed within a patient's brain and configured to detect electrical signals generated from the brain, and a control module in communication with the at least one electrode. The control module may include an event detection device configured to identify occurrence of the neurological event based on the detected electrical signals, and a data recording device including a counter synchronized with an external clock. Upon identifying occurrence of the neurological event by the event detection device, the recording device may be configured to record the detected electrical signals and a value of the counter. The value of the counter may be configured to increase by one in every predetermined time interval.

Another aspect of the invention may include an external device configured to communicate with the control module. The external device may be configured to receive the value of the counter and the detected electrical signals from the remote module. The external device may be configured to convert the value of the counter to a real-time value.

In still another aspect of the invention, the external device may be configured to transmit a start signal to the control module to upload the value of the counter and/or the detected electrical signals to the external device or other processing device. Alternatively or additionally, the external device may be configured to receive a start signal to the remote module or other processing device to download the value of the counter and/or the detected electrical signals from the control module.

In another aspect of the invention, the at least one electrode may include at least one multi-electrode array, and the multi-electrode array may include a plurality of electrodes. The plurality of electrodes may be configured to penetrate into neural tissue of the brain to detect electrical signals generated from the neurons. The at least one electrode may be configured to detect local field potentials of the brain, electrocorticogram (ECoG) signals, and/or electroencephalogram (EEG) signals.

In still another aspect of the invention, a device for placing an implant in a patient's body may be provided. The device may include an elongated member having a distal sleeve, the distal sleeve having a first portion and a second portion and configured to receive the implant between the first portion and the second portion. The first portion and the second portion may be configured to move relative to each other. At least the first portion may include an expandable member so as to push the implant towards an implant site in the patient's body.

In another aspect of the invention, the device may include the implant, wherein the implant may include a plurality of electrodes for placement in a brain of the patient, and wherein at least one of the first portion and the second portion may be configured to cover the plurality of electrodes. In an aspect, the first portion may be inflatable.

In still another aspect of the invention, the device may include a substantially rigid backing member, wherein the first portion may be configured to push against the backing member to expand towards an implant site. In yet still another aspect of the invention, the device may include a grasping member to grasp the implant. In an aspect of the invention, at least a portion of the device may be made of a bioabsorbable material.

Another aspect of the invention may provide a system for detecting occurrence of an undesired activity in a person. The system may include an implant configured to be placed in the body and detect signals indicating that the undesired activity is occurring or is about to occur. The sytem may also include a processing unit configured to process the detected signals and generate a control signal to prevent the undesired activity and/or warn the person or a third person. The control signal may be at least one of an electrical signal and a chemical signal. The control signal may be inputted to the brain. Alternatively or additionally, the control signal may be inputted to at least a portion of the central nervous system and/or peripheral nervous system to prevent the undesired activity.

In still another aspect of the invention, a system for detecting and treating a neurological event in a patient's body may be provided. The system may include an implant configured to be placed in the body and detect signals generated from the body, an external device, and a processing unit configured to process the detected signals and generate a control signal that controls the operation of the external device. The external device may be a movement device, where the movement of the device may be controlled by the processing unit.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

In the drawings:

FIG. 1 is a schematic illustration of a neurological event monitoring and therapy system, according an exemplary embodiment of the invention;

FIG. 2 is a schematic illustration of a remote brain implant, according to an exemplary embodiment of the invention;

FIG. 3 is a detailed perspective view of an exemplary multi-electrode array shown in FIG. 2;

FIG. 4 is a schematic illustration of a remote brain implant, according to another exemplary embodiment of the invention;

FIG. 5 is a schematic illustration of a remote brain implant, according to still another exemplary embodiment of the invention;

FIGS. 6-7 are flow diagrams illustrating various methods of establishing a database for use in, for example, predicting occurrence of a neurological event, according to various exemplary embodiments of the invention;

FIGS. 8-9 are flow diagrams illustrating various methods of adaptive signal processing for use in, for example, predicting occurrence of a neurological event, according to various exemplary embodiments of the invention;

FIG. 10 is a diagram illustrating various biofeedback mechanisms, according to various exemplary embodiments of the invention;

FIGS. 11-12 are schematic illustrations of various external devices used, for example, in various biofeedback mechanisms, according to various exemplary

FIG. 13 is a diagram illustrating various methods for preventing occurrence of neurological events; and

FIGS. 14-15 are schematics illustrations of a device and method for placing an implant in a patient's body, according to an exemplary embodiment of the invention.

### DESCRIPTION OF THE EMBODIMENTS

Reference will now be made in detail to the exemplary embodiments of the invention, examples of which are illustrated in the accompanying drawings. Wherever possible, the same reference numbers will be used throughout the drawings to refer to the same or like parts.

Systems and methods consistent with the invention may detect various neural, biological, or physiological signals generated within a patient's body, and process those signals to predict certain neurological events prior to their occurrence and/or to generate one or more control signals to suppress or control the neurological events. While the invention will be described in connection with a particular epileptic event, the invention may be applied to, or used in connection with, treatment of any other types of sensory or motor disorders, such as, for example, headaches, dizziness, and stroke, numerous neurological or neuropsychiatric disorders, such as, for example, depression, Parkinson's disease, or Alzheimer's disease, various biological conditions, such as, for example, cardiovascular disease, obesity, eating disorders, substance abuse or addiction, obsessive compulsive disorder, schizophrenia, mania, panic attacks, apnea, sleep apnea, other sleep disorders, movement disorders such as tourette's, tics, cerebral palsy, or dystonia, or various biological or physiological activities, such as, for example, voluntary or involuntary criminal or unsocial activities.

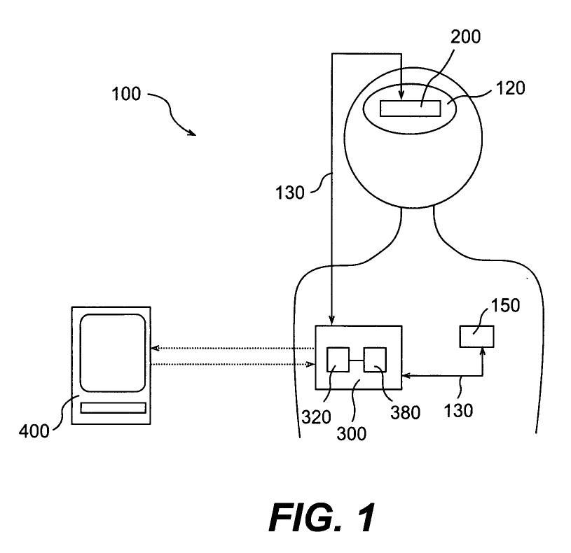

According to an exemplary embodiment of the invention, FIG. 1 illustrates a brain-machine interface (BMI) system 100 for monitoring epileptic activities in a patient's body. The system 100 may detect various signals generated from the body and process these signals to characterize various seizure-inducing conditions, differentiated from normal conditions, to accurately predict a future epileptic seizure or detect a current epileptic seizure. Upon predicting or detecting a seizure, the system 100 may be configured to generate one or more signals that may be used, for example, to suppress or control the seizure. As will be described in detail herein, the prediction and/or detection capability may also provide a warning signal to the patient and/or another individual (e.g., physician or family members), so that the patient or other individual may take appropriate responsive actions to suppress, dampen, or delay the seizure or to eliminate any possible harmful situation that may result from the seizure.

As shown in FIG. 1, the system 100 may include a remote brain implant 200 placed in or on the brain 120 for detecting electrical signals indicative of spatial or temporal neural activities of the brain 120 and a central processing module 300 for processing the detected electrical signals and generating one or more signals for treating the epileptic seizure, such as suppressing, dampening, delaying, or otherwise treating. The system 100 may also include one or more sensors 150 for detecting other biological or physiological activities of the body, such as, for example, muscle movement including tremors, heartbeat rate, skin conductivity, pupil movement or dilation, perspiration, respiration, or levels of one or more blood constituents, such as dissolved oxygen or glucose, brain temperature, pressure, magnetic or electrical conductivity characteristic, which may be used in combination with the detected neural activities in the brain to predict or detect the epileptic seizure. In an exemplary embodiment, the module 300 may include an event detector 320 for detecting certain conditions, which may be characterized as precursory conditions of a seizure, and a data recorder 380 for recording the detected electrical signals characterizing those seizure-inducing, precursory conditions. Moreover, the module 300 may be configured to detect a current seizure and generate a control signal to suppress, dampen, delay, or control the seizure.

The sensor 150 and the brain implant 200 may be connected to the module 300 via suitable connections 130, which may be optical fibers, metallic wires, telemetry, combinations of such connectors, or wireless transceivers, or other conductors or data transceivers known in the art. As will be described further herein, the system 100 may include one or more external devices 400 for receiving, storing, and/or processing information, and/or providing a biofeedback to the patient, e.g., warning of a forthcoming seizure and/or information relating to the patient's condition, so that the patient can take appropriate responsive actions to suppress or control the seizure. The external device may be one or more of visual indicators, auditory indicators, and tactile transducers, such as, for example, a computer, a cell phone, a beeper, or a PDA. Alternatively or additionally, this information may be supplied to a caretaker or clinician. The information may be sent to a local display device or any other suitable remote device known in the art.

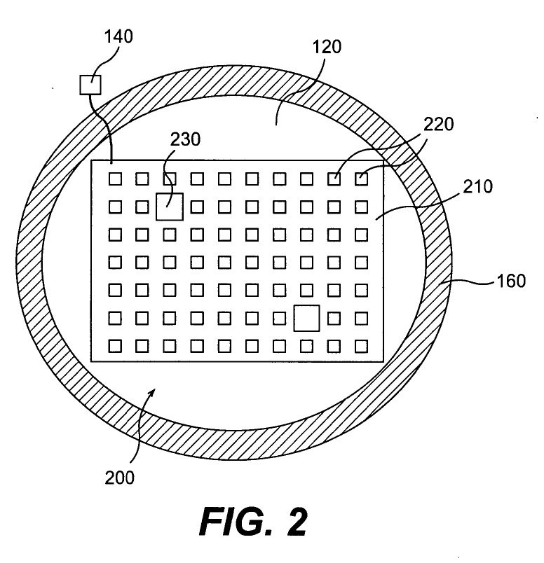

FIG. 2 shows a remote brain implant, or sensor, 200, according to an exemplary embodiment of the invention. The sensor 200 may include a subdural grid 210 having a plurality of rows of electrode contacts 220 configured to contact the cortical surface in the subdural or epidural space of the brain 120. Each electrode contact 220 may be individually connected to a connector 140 and the connector 140 may be connected to the central processing module 300 for processing of the detected electrical signals representative of the neural activity in the brain 120. Alternatively, the subdural grid and multi-electrode arrays may each have individual connectors (not shown). The large area of coverage in the brain 120 by the subdural grid 210 may enable monitoring of the overall neural activity in the brain 120 and may provide information relating to the precise localization of the epileptic focus. The shape or size of the grid 210, as well as the number of electrode contacts 220, or number of separate electrode sensor arrays, may vary depending upon, for example, the geometry and size of the implantation site in the brain 120. In various exemplary embodiments, the subdural grid 210 may include multiplexing circuitry (not shown), e.g. buried in a flex circuit in the subdural grid 210, which may be used to reduce the number of wires extending from the implant 200 through the scalp or to a separate implant. For example, each wire extending from each of the contacts 220 in the subdural grid 210 may be connected to the multiplexing circuitry, which may then multiplex the detected signals in the wires into a reduced number of data lines connected to the module 300. Appropriate demultiplexing circuitry may then be present at the module 300 to demultiplex the received signals to appropriately process the neurological signals detected by the contracts 220. Alternatively or additionally, the multiplexing circuitry may include a preprocessor for preprocessing the detected signals (e.g., discriminating or discretizing the signals) to reduce the amount of information sent to the module 300.

The implant 200 may also include one or more multi-channel, high-density, micro-multi-electrode arrays 230, placed preferably at or near a suspected epileptic focus area or in such a way that seizure onset and spread can be electrically recorded by the array. The arrays 230 may penetrate into the neural tissue of the brain 120 to allow each electrode to record electrical signals, light, and/or acoustic waves generated from one or more neurons in the cortex. In an exemplary embodiment, individual spiking signals may be detected from the cortical surface (i.e. without penetrating). In various exemplary embodiments of the invention, various exemplary arrays disclosed in U.S. Pat. No. 5,215,088 to Normann et al., entitled “Three-Dimensional Electrode Device,” U.S. Pat. No. 6,171,239 to Humphrey, entitled “Systems, Methods, and Devices for Controlling External Devices by Signals Derived Directly from the Nervous System,” and copending U.S. patent application Ser. No. 10/717,924, filed Nov. 21, 2003, by Donoghue et al., entitled “Agent Delivery Systems and Related Methods Under Control of Biological Electrical Signals,” the entire disclosures of which are incorporated by reference herein, may be used in connection with various systems and methods of this invention.

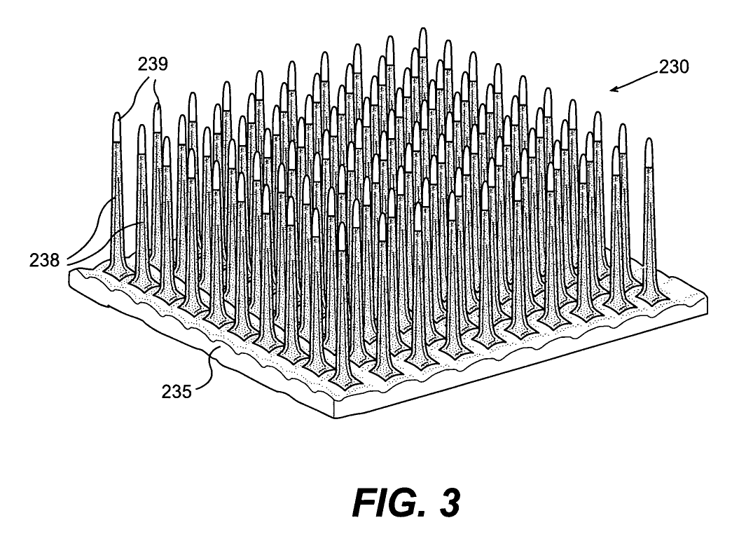

As shown in FIG. 3, the multi-electrode array 230 may include a substrate 235 made of, for example, durable biocompatible material (e.g., silicon), and a plurality of sharpened projections 238 that may project from the substrate 235 and contact with or extend into the brain 120. Each projection 238 may have an active electrode distal tip 239 and may be electrically isolated from neighboring electrodes 239 by a suitable non-conducting material. In an exemplary embodiment, one or more projections 238 may include multiple electrodes 239 along its length. Also, the array 230 may include different types of electrodes, such as, for example, recording electrodes, stimulating electrodes, photo sensors, acoustic transducers, or any combination thereof. Alternatively or additionally, the differences between electrode types may include different materials of construction, coatings, thicknesses, geometric shapes, etc. Each of the recording electrodes 239 may form a recording channel that may directly detect electrical signals generated from each of the neurons in the electrode's vicinity. Further signal processing may isolate the individual neuron signals, each of which may comprise a series of electrical spikes, so as to precisely localize a seizure focus. Alternatively or additionally, while the electrodes 239 may detect multiple individual neuron signals, only a particular subset of the electrodes 239 may be selectively chosen for further processing. A suitable preprocessing method, such as, for example, a calibration process, may be used to selectively choose the subset of the electrodes 239.

In an exemplary embodiment, the array 230 may also include one or more electrodes with a fluid reservoir (not shown) for storage and delivery of therapeutic agents or drugs. For example, an exemplary array disclosed in the above-mentioned copending U.S. patent application Ser. No. 10/717,924 by Donoghue et al., the entire disclosure of which is incorporated by reference herein, may be used in connection with various systems and methods of this invention.

In another exemplary embodiment, the array 230 may be removably arranged with the subdural grid 210, so that the array 230 may be easily repositioned to a different location within the grid 210 to facilitate detection and fine-tuning of the localization of epileptic focus. In an alternative embodiment, the array 230 may be placed or removed independent of the subdural grid 210.

According to an exemplary embodiment of the invention, the combination of the subdural grid 210 and the multi-electrode array 230 provides a unique signal processing capability that may be used, for example: to predict an epileptic seizure prior to its occurrence; to confirm one or more epileptic focus prior to a surgical resection; to find multiple foci; and/or to characterize an epileptic activity. While the subdural grid 120 may provide volume current or voltage potentials of the brain 120, the multi-electrode array 230 can measure each individual neuron's cellular activity and, as a whole, can measure local field potentials (LFPs) and other signals between single neuron and EEG recordings.

Therefore, when the subdural grid 210 is used in combination with the multi-electrode array 230, a variety of new analytical information may become available when those measured values in the subdural grid 210 and the multi-electrode array 230 are combined and visualized at different signal levels. For example, the time/space interactions between the above-mentioned volume current potentials, individual cellular activity, and LFPs may present new computational and/or signal processing methods that may enable prediction of a particular epileptic event. For example, the following exemplary array of detected neuron potentials (shown only in part) may be generated at the single cell level:

$$( \begin{matrix}1 & 0 & 1 & 1 & 1 \\ 1 & 1 & 1 & 1 & 1 \\ 1 & 1 & 0 & 1 & 1 \\ 1 & 0 & 1 & 1 & 1 \\ 1 & 0 & 1 & 1 & 1\end{matrix} )$$

where “1” represents a firing neuron. These values may be substituted with other measures of a cell activity or a vector of activity. In an epileptic tissue, the implant 200 may then observe and characterize a stereotyped, predictable pattern with a set of rules that may describe how neighboring neurons affect each other (i.e., cellular automata or correlation index). Based on these characterized patterns or models, it may be possible to predict epileptic events because, at the next signal level (e.g., at LFP level), it may be possible to derive a partial differential equation that may describe the time and space evolution of the cellular activity. For example, certain epileptic events may be described using the following equation:

$$\frac{{\partial }^{n} V ( r , t )}{\partial {r}^{n}} = K \frac{{\partial }^{m} V ( r , t )}{\partial {t}^{m}} ,$$

where V(r,t) is the measured voltage at position r and time t. The constant K may contain detailed information relating to, for example, evolution of firing patterns in time and space, which may be used to describe and characterize, for instance, how each of the neurons in the focus area interacts with its neighboring neurons and how its behavior evolves in time.

Based on these characterized evolutional cellular behavior, it may be possible to correctly predict a future occurrence of an epileptic event and the timing of that event. For example, with this new set of information, the detected and recorded electrical signals from large populations of neurons may be reanalyzed using various analytical methods to further characterize and define, for example, the epileptic focus and/or its behavior. Once sufficient information is gathered, which characterizes the epileptic focus and/or its behavior leading up to epileptic seizure, that information may be stored in a database, with which newly detected signals may be compared, to predict or detect occurrence of an epileptic seizure. For example, a suitable sensor may be placed in the vicinity of the focus to detect various signals. The detected signals may then be compared with those stored in the database to determine whether the detected signals include one of the signals characterizing occurrence of the epileptic seizure. Alternatively or additionally, the detected signals may be compared with any other suitable target signals, such as, for example, a target look-up table, neural nets, or a Bayesian probabilistic framework.

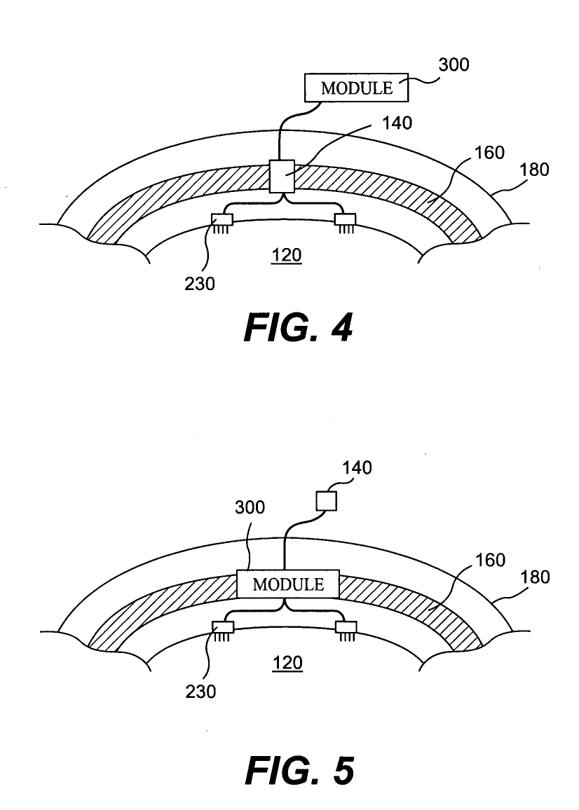

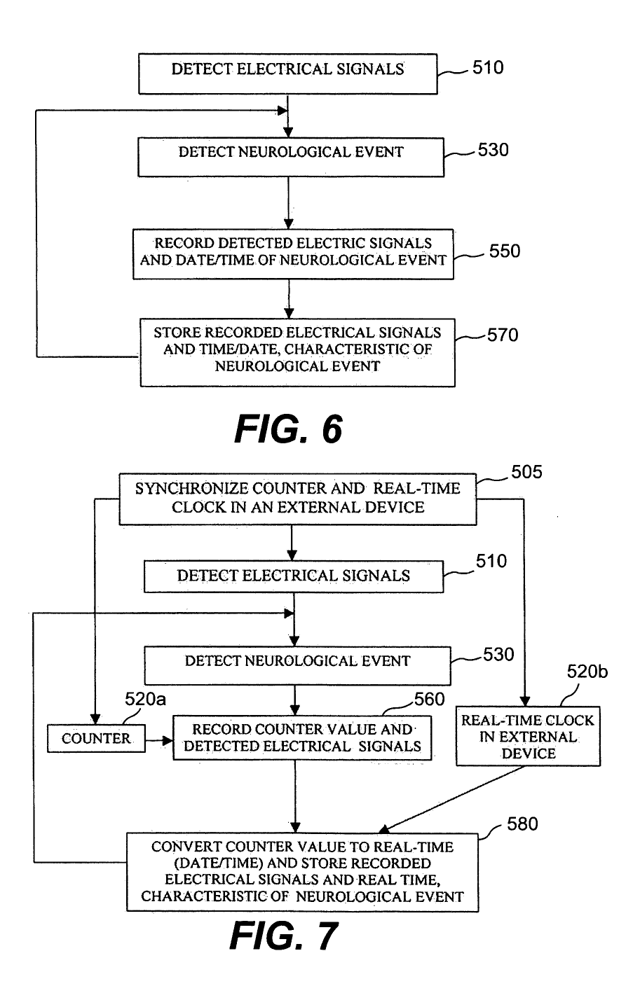

In an alternative embodiment, the brain implant 200 may include one or more multi-electrode arrays 230 without the presence of a subdural grid, as shown in FIGS. 4 and 5. The arrays 230 may be placed at, or in the vicinity of, the suspected epileptic focus or at a location where a neural activity having an identifiable pattern of a seizure-inducing condition is likely to occur. In various exemplary embodiments, the implant 200 may include three or more multi-electrode arrays 230 so that triangulation signal processing and signal location techniques, similar to that used in target positioning systems, may be used to locate, or otherwise characterize one or more epileptic foci.

Prior to the placement of the arrays 230, a subdural grid and/or other suitable detection or imaging devices and methods may be used to identify a target location of the seizure focus. In an exemplary embodiment, a subdural grid may be initially used to localize the epileptic focus and then be removed from the brain 120, leaving or placing the multi-electrode arrays 230 in the brain 120 at the suspected epileptic focus. In this case, an ambulatory device or an implanted device may be attached to the arrays 230, so as to enable communication with, for example, an external device for two-way information transfer.

According to another exemplary embodiment of the invention, the implant 200 may include other suitable invasive or noninvasive sensors that may sense electrical signals from the brain 120. For instance, the implant 200 may include non-penetrating or noninvasive sensors, such as one or more multi-channel electroencephalogram (EEG) sensors, placed on the surface of the scalp and/or any other invasive or noninvasive sensor, which may obtain information in the form of neuron spikes, local field potentials (LFPs), or electrocorticogram signals (ECoGs). In any event, the implant 200 and/or the system 100 may be configured to sense or detect other forms of electrical information, or combinations of types of electrical information, depending on, among other things, the type and resolution of the desired information. For example, the system 100 may include other electrodes, such as, for example, scalp electrodes, wire electrodes, and cuff electrodes, which may be placed throughout the central nervous system or various parts of the patient's body. These electrodes (not shown) may be configured to interact with the brain implant 200 and the central processing module 300. In an exemplary embodiment, the system 100 may include a movement sensor (e.g., strain gauge) or a pressure monitoring device (e.g., a differential pressure transducer) placed in the brain 120 to detect contraction of the brain 120, which may precede an epileptic seizure. The contraction of the brain 120 may cause a slight differential pressure within the brain 120 or movement of the brain 120, which may be detected by the movement sensor or the pressure monitoring device. In still another exemplary embodiment of the invention, the system 100 may include a spectrophotometer or any other suitable optical device to measure the change in optical density, which may precede an epileptic seizure, and the resulting signals may be characterized as a predictive parameter for predicting a seizure activity. Alternatively or additionally, the implant 200 or any other sensor may be configured to monitor the changes in temperature, pH, or magnetic resonance intensity, which may precede an epileptic seizure.

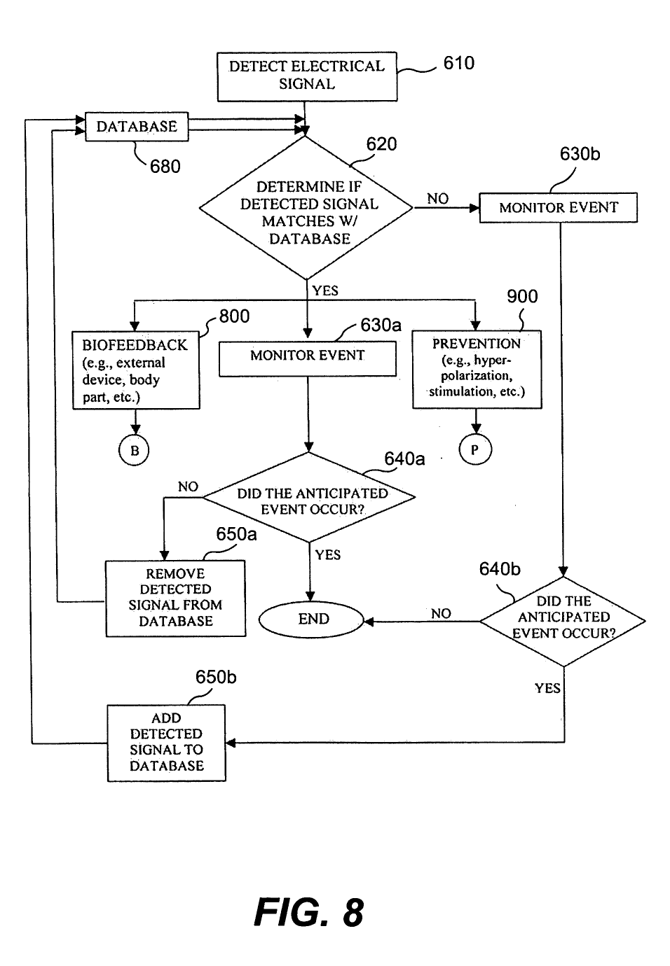

The module 300 may be implanted within or on a patient's body, such as, for example, the brain 120 or the abdomen. In an exemplary embodiment, the module 300 may be placed in, on or under the patient's skull 160, as shown in FIG. 5, but it may be placed on or in any other portion of the body, such as scalp 180, chest area, abdomen, or neck, or the device could be external and unattached to the body. In another exemplary embodiment, the module 300 may be configured to be attachable to the patient's body or clothing. The central processing module 300 may process the detected electrical signals indicative of neural activities to perform various functions, including, but not limited to, receiving, recording, monitoring, displaying, and/or transmitting electrical signals, processing the electrical signals to create one or more control or biofeedback signals, transmitting the control signals, and/or sending or receiving power to or from the implant 200 or other sensors 150. Each of the various processes carried out by the module 300 in connection with the implant 200, sensor 150, or one or more external devices will be described in detail with reference to FIGS. 6-13.

FIGS. 6 and 7 illustrate various methods for detecting a neurological event in a patient's brain and establishing a database for predicting or detecting occurrence of the neurological event, according to various exemplary embodiment of the invention. The neurological events may be characterized by electrical signals generated within the patient's brain and the methods consistent with the invention may detect those electrical signals (step 510) to characterize a particular neurological event or a precursory condition to such an event (step 530).

The processing module 300 may preprocess the received electrical signals before processing the signals for extraction of neural information. The preprocessing may include, but not limited to, measuring the background signals and calibrating the detected signal based on the measured background signals, noise filtering, impedance matching, rectifying, integrating, differentiating, discretizing, and amplifying the signals. In addition, the module 300 may characterize the obtained neural signals in comparison with the other biological and/or physiological signals and differentiate abnormal neural signals from those resulting from normal activities, such as, moving arms. The module 300 may employ a neuron separation algorithm to sort neural spikes and/or a spatial differentiation algorithm to spatially differentiate signals from the same multi-electrode array.

In an exemplary embodiment shown in FIG. 6, when the neurological event is detected (530) based on the detected electrical signals (510), the central module 300 may transmit a signal to a recording device to record the detected electrical signals together with the timing information at which the neurological event took place (step 550). The recorded electrical signals and the timing may then be stored in a storage or memory device tor use in a future referencing procedure (step 570).

In an alternative embodiment shown in FIG. 7, the module 300 or the implant 200 may not include a real-time clock. Instead, the system 100 may include a simple counter 520a in the implant 200 or the module 300, that may be synchronized with a real-time clock 520b placed in an external device (step 505). For example, the counter may be configured to increase its value by one unit in every 30 seconds of real-time, such that the counter value may be directly convertible into a time of day and date value at a later time by a separate device. One of the advantages of using such a counter system may be to simplify the design or reduce the power requirements of the module 300 or the implant 200 by eliminating the real-time clock.

Therefore, as shown in FIG. 7, prior to detection of the electrical signals, the counter 520a and the real-time clock 520b may be synchronized (step 505) to a reference point in time. Thereafter, the electrical signals may be detected (step 510). When the neurological event is detected, the central module 300 may transmit a signal to a recording device to record the detected electrical signals together with the value of the counter 520a (step 560). The recorded counter value may then be converted into a real-time value (e.g., time/date), and the converted real-time value may be stored together with the recorded electrical signals in a storage or memory device for use in a future referencing procedure (step 580).

The stored electrical signals may then constitute a database for future reference which may be used to predict occurrence of the neurological event. The database may be further processed, for example, by adaptive signal processing to identify and characterize the predictable pattern of neuron activity or a precursory condition that may precede the neurological event.

According to an exemplary embodiment of the invention, the processing module 300 may provide a unique signal processing technique utilizing an adaptive processing mechanism. For example, the processing module 300 may conduct adaptive processing of the detected electrical signals indicative of a predictable neurological event by changing one or more parameters of the system to improve the predictive performance.

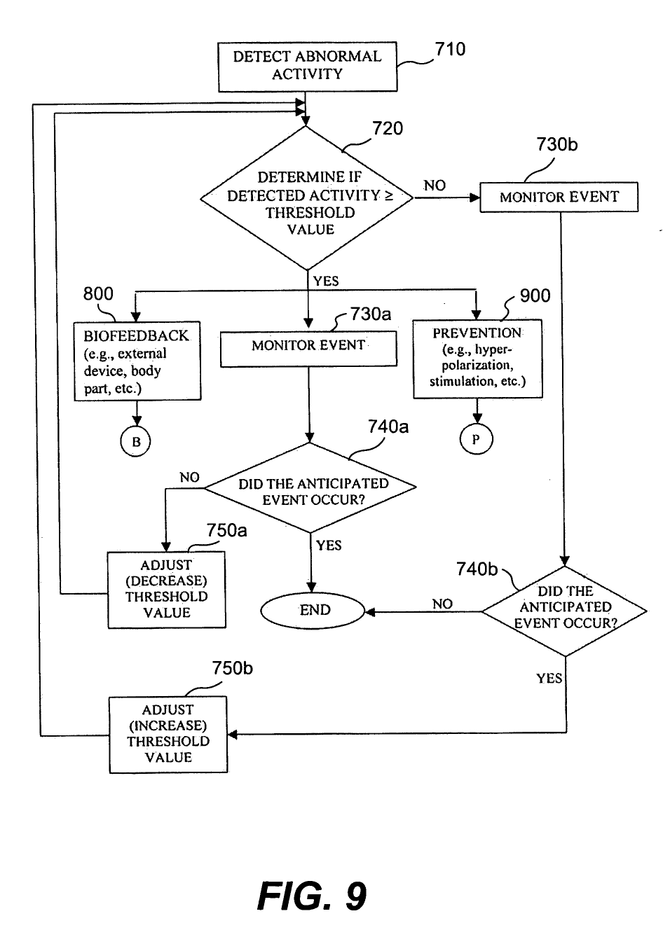

FIG. 8 illustrates an exemplary adaptive signal processing method according to an embodiment of the invention. First, electrical signals indicative of abnormal activity in the brain 120 or other parts of the body may be detected by, for example, the brain implant 200 and/or other sensors 150 (step 610). These detected electrical signals may then be compared with the pre-collected or otherwise identified target signals (i.e., characterizing the condition for occurrence of the event) stored in a database 680 to determine whether the detected signals substantially match with any of the target signals stored in the database (step 620).

Regardless of whether the detected signals match with one of the predictive conditions, the system 100 may allow continued monitoring and detecting of electrical signals from various parts of the body (step 630a, 630b) to determine whether the anticipated event actually occurred or not (step 630a, 630b and step 640a, 640b). Depending upon the actual occurrence of the anticipated event, the database may be modified or adjusted to correct the mistaken prediction of the event. For example, if step 620 has predicted that the event would occur, but the event did not actually occur, the database 680 may be modified to remove the set of target signals corresponding to the detected signals from the database (steps 650a and 680). By the same principle, if step 620 did not predict the event, but the event actually took place, the database may be modified or adjusted to add the detected signals to the database as additional target signals (steps 650b and 680). Therefore, the longer the system 100 is in operation, the more accurate target data can be accumulated, thereby reducing the mistaken prediction. In a preferred embodiment, the system 100 may be biased to reduce the number of false negative predictions, favoring predicting a seizure that does not occur over missing an actual seizure. Alternatively or additionally, the system 100 may include an external device that a user (e.g., clinician) may manually create and/or modify the database. For example, the clinician may observe a patient's condition with respect to an epileptic seizure and, based on the observation, may modify the database to reduce the mistaken prediction in the future.

FIG. 9 illustrates another exemplary adaptive signal processing method, according to another embodiment of the invention. The basic operational principles of this embodiment may be substantially identical to the embodiment described with respect to FIG. 8, except that, in this embodiment, a predetermined threshold value may be used to differentiate the predictive signals or pattems preceding the neurological event from other conditions resulting from normal activities. For example, as shown in FIG. 9, the system 100 may detect signals or parameters indicative of an abnormal activity in the brain 120 or other parts of the body (step 710). These detected signals or parameters may then be compared with the predetermined target threshold value to predict whether the neurological event would occur or not (step 720). Again, regardless of the prediction outcome, the system 100 may continue to monitor various signals or parameters (steps 730a, 730b) to determine whether the anticipated event actually occurred or not (step 730a, 730b and step 740a, 740b). Depending upon the actual occurrence of the anticipated event, the target threshold value may be modified or adjusted to correct the mistaken prediction of the event. For example, if step 720 has predicted that the event would occur, but the event did not actually occur, the target threshold value may be adjusted (e.g., decreased), as depicted in step 750a. On the other hand, if step 720 has not predicted occurrence of the event, but the event actually took place, the threshold value may also be adjusted (e.g., increased), as depicted in step 750b, to fine tune the differentiating factors between the predictive neurological event and the other normal activities (step 750b).

Alternatively or additionally, various exemplary embodiments of the adaptive signal processing may include, but not be limited to, changing a parameter during a system calibration, changing a method of encoding neural information, changing the type, subset, or amount of neural information that is processed, or changing a method of decoding neural information. Changing an encoding method may include changing neuron spike sorting methodology, calculations, or pattern recognition. Changing a decoding methodology may include changing variables, coefficients, algorithms, constants such as offset bias, and/or filter selections. Other examples of adaptive processing may include changing over time the type or combination of types of signals processed, such as EEG, LFP, neural spikes, or other signal types. U.S. Pat. No. 6,171,239 to Humphrey and entitled “Systems, Methods, and Devices for Controlling External Devices By Signals Derived Directly From the Nervous System,” the entire disclosure of which is incorporated by reference herein, discloses adaptive processing methodology that may be used in connection with various systems and methods of this invention.

In accordance with another embodiment of the invention, as shown in FIGS. 8 and 9, the system 100 may generate one or more biofeedback signals (step 800) to provide the patient, another individual, and/or an external device with information relating to the patient's condition with respect to the anticipated neurological event. For example, once a neurological event is predicted, the module 300 may itself display the patient condition or communicate with a suitable external device 400, to which the patient has an immediate access, via a wired or wireless connection, to inform the patient that a certain neurological event is about to occur. For example, if a PDA (i.e. a personal digital assistant) is used as the external device, the PDA screen may display a sign indicative of, for example, the time, likelihood, and severity of forthcoming seizure.

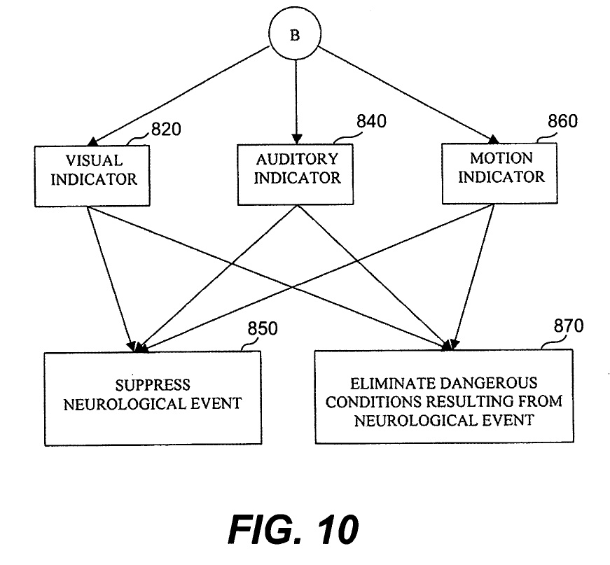

Referring to FIG. 10, such an external device 400 may provide a warning signal to the patient (steps 820, 840, 860), so as to give the patient an opportunity to suppress the neurological event (step 850 of FIG. 10), self-medicate, seek care, or to eliminate potentially dangerous conditions that may result from the neurological event (step 870). As shown in FIG. 10, various devices and methods may be used to serve these purposes. For example, in an exemplary embodiment, the patient's condition with respect to the neurological event may be displayed visually on a visual indicator including, but not limited to, a computer monitor, a cell phone, a patient worn device such as a wrist worn display, or a PDA. Alternatively, a tactile transducer, a sound transducer, or any other type of transducer known in the art may be worn by the patient. The transducer may be activated when an event is predicted or detected.

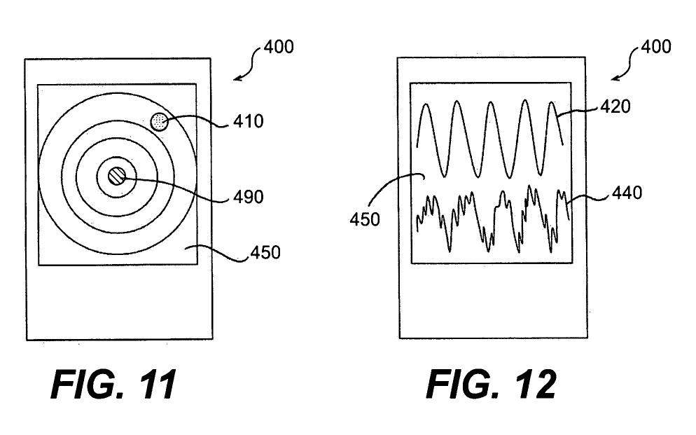

FIGS. 11 and 12 show various exemplary contents that may be displayed in the visual indicator 400. The embodiment shown in FIG. 11 displays a dot 410 on a screen 450 of the visual indicator 400, which may indicate the deviation of the patient's condition from a normal target condition displayed, for example, with another dot 490 at the center of the screen 450. The distance between the patient's dot 410 and the target dot 490 may indicate the severity of the patient's condition. Other suitable marks, such as arrows or lines, may also be used. In another exemplary embodiment, the patient's condition may be displayed in a waveform 440 in comparison with a healthy target waveform 420, as shown in FIG. 12. The indicator 400 may also display the time remaining until the occurrence of the neurological event. The indicator 400 may also display the probability of the neurological event occurring and the predicted severity of the neurological event. In another exemplary embodiment, the indicator 400 may provide instructions for effectively preventing, delaying, or diminishing the neurological event. Alternatively or additionally, the module 300 may transmit a target or healthy current waveform to the implant 200 or other sensors 150, so as to change activity or bring about lasting neuroplastic changes that prevent the neurological event.

An important advantage of having the patient's condition displayed on a visual indicator 400 in comparison with a target condition is that the patient has an opportunity to take appropriate actions to bring the patient's condition close to the healthy target condition, thereby suppressing the neurological event from occurring. For example, while observing the visual indicator 400, the patient may try various activities and/or learn by trial-and-error one or more activities that may effectively bring the patient's mark close to the healthy target mark. In this manner, the patient may be trained to respond rather quickly to suppress the unwanted neurological event.

In another exemplary embodiment, the biofeedback may be provided to an auditory indicator (step 840 of FIG. 10), such as a beeper. The operation of the auditory indicator may be similar to that of a visual indicator to the extent that the auditory indicator may produce a variety of distinct sounds, indicative of the time remaining and/or deviation from the healthy target condition. For example, in an exemplary embodiment, the auditory indicator may generate a variety of different beeping sounds with different intervals or sounds with different pitch, tone, and/or volume.

In another exemplary embodiment, as a variation of the auditory indicator (step 840 of FIG. 10), the system 100 may directly stimulate an area of the brain related to auditory perception via, for example, an electrical current or chemical injection. The examples of such an area may include, but be not limited to, the organ or Corti, vestibulocochlear cranial nerve, cochlear nuclei, trapezoid bodies, inferior colliculi, medial geniculate nuclei, primary and secondary auditory cortices, planum temporale, Wemicke's area, and higher order parietal cortices.

In other various exemplary embodiments, the biofeedback equipment may include light emitting devices, such as devices that flash light as an indicator, and/or tactile transducers, such as force transducers, heating or cooling transducers, olfactory transducers, and electric shock transducers. In these exemplary embodiments, various feedback mechanisms, such as, for example, volume, frequency, temperature, and force, may correlate to time remaining until a seizure occurrence, severity, likelihood, type of seizure, and/or other various seizure parameters.

In still another exemplary embodiment, the system 100 may transmit, as a biofeedback to the patient, an energizing signal to the implant 200 to cause an involuntary, yet continuous, movement of a specific portion of the body (step 860), such as ticking a finger or toe. The energizing signal may stimulate one or more brain cells coordinating movement of the specific body portion, causing involuntary movement of that portion, so as to give notice to the patient that a neurological event is about to occur, and avoiding the need for a separate external device that is wom or carried by the patient. Similar to the other exemplary methods discussed above, upon noticing the involuntary movement of a body portion, the patient may take appropriate actions to stop the involuntary movement and suppress the neurological event, or to avoid potentially dangerous situations by, for example, pulling the car over to side of road, if the patient was driving, lying down on a bed, or getting out of a shower, until the event passes by. Alternatively or in addition, the module 300 may transmit a control signal to the implant 200 for delivery of a drug or other therapeutic agent to suppress the neurological event. In an exemplary embodiment, a drug delivery system described in a copending U.S. patent application Ser. No. 10/717,924, the entire disclosure of which is incorporated by reference herein, may be used. In another embodiment, the visual reporting mechanism described herein may be affected by stimulating, through, for example, depolarizing or hyperpolarizing electrical current, some part of the brain related to visual function, such as the primary or secondary visual cortices, inferotemporal cortex, fusiform cortex, optic nerves, optic chiasm, optic lateral geniculate nuceli, optic radiation, superior colliculus, higher order parietal cortices, and/or frontal eye field cortices.

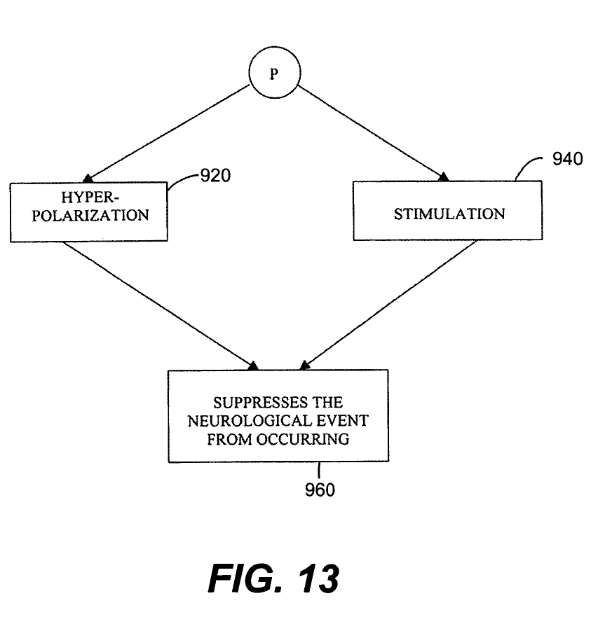

As shown in FIGS. 8 and 13, the system 100 may alternatively or additionally generate one or more control signals which may be used to suppress occurrence of the neurological event (steps 900 and P). According to an exemplary embodiment of the invention, once a forthcoming neurological event is predicted or detected (step 620 of FIG. 8 or step 720 in FIG. 9), the system 100 may automatically generate a stimulating signal (e.g., electrical or chemical signal) which may be transmitted to the implant 200 in the brain (e.g., to stimulating electrodes in the implant 200), the central nervous system, or other various parts of the patient's body (step 940), to suppress the neurological event (step 960). In an exemplary embodiment, a stimulating electrical current may be sent to the neural focus area in the brain to cause the neurons to overfire or become refractory, thereby suppressing the neurological event.

In another exemplary embodiment, once a forthcoming neurological event is predicted or detected (step 620 of FIG. 8 or step 720 in FIG. 9), the system 100 may automatically generate a hyperpolarizing signal to the brain 120 to hyperpolarize at least a portion of the brain 120 (step 920), thereby suppressing the neurological event (step 960). In an exemplary embodiment, a DC bias current may be sent, as a hyperpolarizing signal, to the neural focus region in the brain 120 to hyperpolarize the neurons and prevent the neurons in that region from integrating synaptic input and firing. In another exemplary embodiment, the neurons may be silenced or manipulated by directing transient stimuli or pulse trains that may change their firing properties, or by polarizing the neurons. In this embodiment, the hyperpolarizing signals may hold the neurons in a non-responsive state.

Alternatively or additionally, the system 100 may depolarize the neurons. With repeated and/or sustained depolarization, the neurons may go into a deep depolarization block because, for example, Sodium channels need to be hyperpolarized, or “cocked” as in a gun before they can promote firing again. Sustained depolarization may prevent the recocking of the Sodium channels. In an alternative embodiment, the control signal may include electromagnetic flux that may induce electrical current in at least a portion of the brain. In another exemplary embodiment, the control signal may inject a time-varying electrical current that includes both depolarizing and hyperpolarizing current into at least a portion of the brain. In still another exemplary embodiment, the control signal may inject a stochastic current pattern into at least a portion of the brain.

In still another exemplary embodiment, one or more electrodes in the implant 200 may have the impedance between those electrodes reduced or shorted in an attempt to prevent, dampen, or delay the neurological event. This reduction in impedance, normally very high, may cause the signals at neighboring neurons to approach one another, thereby preventing large differences between the neurons.

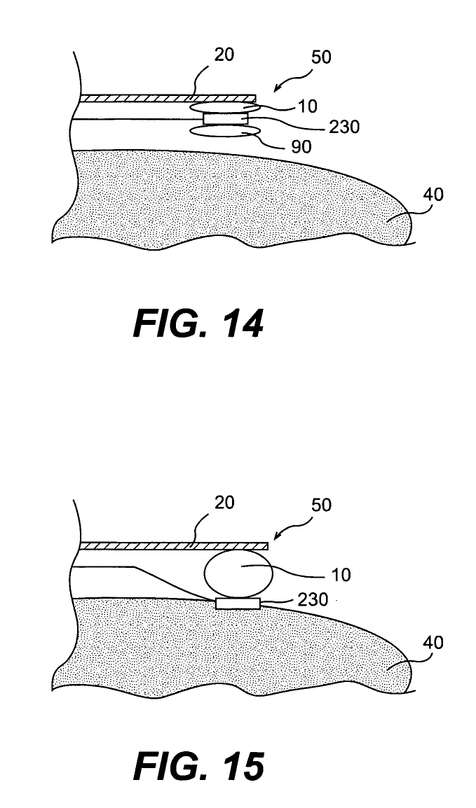

According to another aspect of the invention, devices and methods for placing an implant in a patient's body may be provided. While an exemplary embodiment consistent with the invention will be described with reference to FIGS. 14 and 15, in connection with placement of a particular multi-electrode array in a brain, the invention may be used to place any other type of implant or sensor in any other part of the brain or body.

As shown in FIG. 14, an exemplary embodiment of a device for inserting and placing an implant 230 includes a flexible, elongated shaft having a distal sleeve 50 that may substantially enclose the implant 230 to protect tissues and/or organs in the insertion pathway. The device may be useful in inserting the implant 230 in areas where direct access may not be readily available. For example, much of the cortex in a human is located within sulci or on the mesial surface of the temporal lobe, which may be hidden from normal surgical views or otherwise require angled insertion forces. Since epileptic foci are commonly found in or around these hidden structures, the device shown in FIGS. 14 and 15, having a capability to traverse (e.g., bending or turning) through tortuous paths within the brain, may be used to insert the implant 230 into the area where direct access may not be readily available.

The sleeve 50 may include an upper portion 10 and a bottom portion 90, and may be configured to receive the implant 230 therebetween. At least the upper portion 10 may be expandable, and the upper portion 10 and the bottom portion 90 may be axially movable relative to each other. In an exemplary embodiment, the upper portion 10 may include an inflatable balloon, but other suitable expandable mechanisms, such as, for example, mechanisms utilizing expandable or deformable materials (e.g., shaped memory alloys or polymers), may also be used. In another exemplary embodiment, the upper portion 10 may include a movement-causing member, such as, for example, an electromagnetic actuator (e.g., solenoid), a hydraulic actuator, or a pneumatic actuator. The sleeve 50 may also include a substantially rigid backing surface 20, and the upper portion 10 may push against the backing surface 20 to expand towards a desired placement site 40.

In operation, the sleeve 50 may be inserted, or coupled to a suitable insertion device to guide the sleeve 50, to the desired placement site 40 (e.g., the cortex of the brain 40). Once the implant 230 in the sleeve 50 is properly positioned, the bottom portion 90 may be retracted proximally or extended distally so as to reveal the implant 230, as shown in FIG. 15. The upper portion 10 may then, preferably in a substantially simultaneous action with the retraction or extension of the bottom portion 90, expand its volume or a distance from the backing surface 20 to push the implant towards the desired placement site. Alternatively or additionally, the upper portion 10 may include a grasping member (not shown) to grasp the implant 230.

Once the implant is properly placed, the sleeve 50 may be withdrawn from the body. In an exemplary embodiment, the sleeve 50 may be made of a bioabsorbable material, so that the sleeve 50 may be left in the body to dissolve away without the need for withdrawal.

In another exemplary embodiment, the upper and bottom portions 10, 90 may each include an expandable member. In operation, while the upper and bottom portions 10, 90 are in their balanced expanded state, the bottom portion 90 may be quickly removed or deflated, so as to cause the upper portion 10 to push the implant towards the desired placement site.

Since the placement of, for example, the multi-electrode array in a brain (e.g., on or within a gyrus anywhere in the brain, next to or within a sulcus anywhere in the brain, or a mesial surface of the temporal lobe) may require extreme care and precision, the exemplary devices and methods consistent with the invention as described above may provide a simple and substantially noninvasive way of implanting the array with ensured safety of the patient.

Still another exemplary embodiment of the invention may provide a method of preventing an undesired activity, such as, for example, criminal activity. In this embodiment, the implant 230 may be placed in a “planning” portion of a brain to detect the onset of readiness potentials that may lead to unwanted behavior or activity. Once such behavior or activity is detected, the system 100 may be configured to perform various tasks including, but not limited to, providing a warning signal to the patient or a third party or automatically transmitting electrical and/or chemical input signals to the brain, central nervous system, and/or peripheral nervous system to prevent the unwanted behavior.

According to still another exemplary embodiment of the invention, the system 100 may be combined with an external device, such as, for example, a computer or prosthetic limb, movement or operation of which may be controlled by the system 100. Various other exemplary external devices may include, but not be limited to, a computer display, a mouse, a cursor, a joystick, a personal data assistant, a robot or robotic component, a computer controlled device, a teleoperated device, a communication system, a vehicular system such as a wheelchair or a car, an adjustable bed, an adjustable chair, a remote control device, a Functional Electrical Stimulator device, an artificial limb, a movement assist device, a medical therapeutic equipment such as a drug delivery apparatus, and a medical diagnostic equipment.

Such combination of a BMI and an external device may be useful in a patient having one or more neurological disorders that may accompany disability condition, such as, for example, spinal chord injury or missing limb. The combination may also be used to counteract any neurological events that are caused by or otherwise resulted from the BMI for external device control.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A system for predicting occurrence of a neurological event in a patient's body, comprising:
an implant configured to be placed in the body and detect signals indicative of an activity that precedes the neurological event; and
a processing unit configured to process the detected signals so as to predict the neurological event prior to the occurrence.

2. The system of claim 1, wherein the implant is configured to be placed in a patient's brain.

3. The system of claim 2, wherein the implant includes at least one multi-electrode array, the multi-electrode array including a plurality of electrodes.

4. The system of claim 3, wherein the plurality of electrodes are configured to penetrate into neural tissue of the brain to detect electrical signals generated from the neurons.

5. The system of claim 3, wherein the multi-electrode array includes at least one of a recording electrode, a stimulating electrode, and an electrode having recording and stimulating capabilities.

6. The system of claim 3, wherein the at least one multi-electrode array is configured to detect electrical signals indicative of a neural activity preceding the neurological event.

7. The system of claim 2, wherein the implant is configured to detect electrical signals generated from the neurons located proximate the implant.

8. The system of claim 7, wherein the processing unit is configured to convert the detected electrical signals into a recognizable pattern.

9. The system of claim 8, wherein the recognizable pattern includes a formula describing a behavior of the neurons in time and space.

10. The system of claim 7, wherein the implant is configured to isolate individual neuron signals from neighboring neuron signals.

11. The system of claim 7, wherein the detected electrical signals generated from the neurons include electrical spikes.

12. The system of claim 11, wherein the processing unit is configured to characterize a pattern of the electrical spikes that represent a neural activity preceding the neurological event, so as to predict the occurrence of the neurological event.

13. The system of claim 2, wherein the implant is configured to be placed proximate a neural focus in the brain that initiates the neurological event.

14. The system of claim 2, wherein the implant is configured to detect local field potentials of the brain.

15. The system of claim 2, wherein the implant is configured to detect electrocorticogram (ECoG) signals.

16. The system of claim 2, wherein the implant is configured to detect electroencephalogram (EEG) signals.

17. The system of claim 2, wherein the implant is configured to detect DC potentials.

18. The system of claim 2, wherein the implant is configured to detect light generated from the neurons located proximate the implant.

19. The system of claim 2, wherein the implant is configured to detect acoustic waves generated from the neurons located proximate the implant.

20. The system of claim 2, wherein the implant comprises a subdural grid having a plurality of electrode contacts and configured to be placed on a surface of the brain.

21. The system of claim 20, wherein the implant further comprises at least one multi-electrode array.

22. The system of claim 2, wherein the implant includes a movement sensor configured to detect movement of the brain.

23. The system of claim 2, wherein the implant includes a pressure monitoring device for monitoring pressure in the brain.

24. The system of claim 2, wherein the implant includes a temperature monitoring device for monitoring temperature in the brain.

25. The system of claim 2, wherein the implant includes a magnetic resonance monitoring device for monitoring magnetic resonance intensity in the brain.

26. The system of claim 1, wherein the processing unit is configured to characterize the signals that represent the activity preceding the neurological event.

27. The system of claim 1, further comprising a storage device for storing the signals that represent the activity preceding the neurological event.

28. The system of claim 27, wherein the processing unit is configured to compare the detected signals with the signals stored in the storage device.

29. The system of claim 1, wherein the processing unit includes a recording device for recording the detected signals.

30. The system of claim 1, wherein the implant is configured to detect biological or physiological signals generated within the patient's body.

31. The system of claim 1, further comprising a sensor for detecting other signals generated from the body, the sensor is configured to communicate with the processing unit.

32. The system of claim 31, wherein the processing unit is configured to compare the signals detected by the implant and the other signals detected by the sensor.

33. The system of claim 1, wherein the processing unit is configured to differentiate the signals indicative of the activity that precedes the neurological event from signals resulting from normal activities.

34. The system of claim 1, wherein the processing unit is configured to output information relating to a patient's condition with respect to the neurological event.

35. The system of claim 34, wherein the processing unit includes an indicator for conveying the information to the patient.

36. The system of claim 34, further comprising an external device being in communication with the processing unit, the external device configured to display the information relating to the patient's condition with respect to the neurological event.

37. The system of claim 36, wherein the processing unit is configured to receive an input signal from the external device.

38. The system of claim 36, wherein the external device includes at least one of a visual indicator, a tactile transducer, an auditory indicator, and a light emitting device.

39. The system of claim 34, wherein the information includes a warning signal that the neurological event is expected to occur.

40. The system of claim 34, wherein the information includes a time remaining until the occurrence of the neurological event.

41. The system of claim 34, wherein the information includes an occurrence probability of the neurological event.

42. The system of claim 34, wherein the information includes severity of the neurological event.

43. The system of claim 34, wherein the information includes a patient's current condition in comparison with a normal target condition.

44. The system of claim 34, wherein the information includes instructions for preventing the neurological event from occurring.

45. The system of claim 34, wherein the information includes a stimulating signal provided to the patient to cause a movement of a portion of the patient's body.

46. The system of claim 45, wherein the stimulating signal is sent to the implant.

47. The system of claim 45, wherein the portion of the patient's body is a finger.

48. The system of claim 1, wherein, upon predicting the occurrence of the neurological event, the processing unit is configured to generate a control signal to suppress, dampen, or delay the neurological event.

49. The system of claim 48, wherein the control signal includes an electrical current sent to a patient's brain to stimulate at least a portion of the brain.

50. The system of claim 48, wherein the control signal is configured to stimulate a central nervous system and/or a peripheral nervous system.

51. The system of claim 48, further comprising a drug delivery system, wherein the processing unit sends a signal to the drug delivery system to deliver a therapeutic agent or drug to at least a portion of the patient's body.

52. The system of claim 1, wherein, upon predicting the occurrence of the neurological event, the processing unit is configured to hyperpolarize at least a portion of the brain.

53. The system of claim 44, wherein the processing unit sends a DC bias current to a patient's brain to hyperpolarize the at least a portion of the brain.

54. The system of claim 1, wherein:
the implant includes one or more electrodes; and
upon predicting the occurrence of the neurological event, the processing unit is configured to reduce the impedance between the one or more electrodes.

55. The system of claim 1, further comprising a storage device containing a target signal indicative of the activity that precedes the neurological event.

56. The system of claim 66, wherein the target signal includes a database containing a set of previously detected signals indicative of the activity that precedes the neurological event.

57. The system of claim 66, wherein the processing unit is configured to compare the detected signals with the target signal.

58. The system of claim 66, wherein the processing unit is configured to modify the target signal.

59. The system of claim 1, wherein the neurological event is an epileptic symptom.

60. The system of claim 51, wherein the implant is placed proximate an epileptic focus of a brain.

61. The system of claim 1, wherein the neurological event is an undesired activity.

62. The system of claim 61, wherein the undesired activity includes a criminal activity.

63. The system of claim 61, wherein the implant is configured to be placed in a brain and measure readiness potential of the brain, indicative of occurrence of the undesired activity.

64. A method for treating a neurological event in a patient, comprising:
placing an implant in the patient's body;
detecting signals indicative of an activity that precedes the neurological event; and
predicting occurrence of the neurological event based on the detected signals.

65. The method of claim 64, further comprising placing the implant in the patient's brain.

66. The method of claim 65, wherein the remote implant includes at least one multi-electrode array, the multi-electrode array including a plurality of electrodes.

67. The method of claim 66, wherein the plurality of electrodes are configured to penetrate into neural tissue of the brain to detect electrical signals generated from the neurons.

68. The method of claim 66, wherein the multi-electrode array includes at least one of a recording electrode, a stimulating electrode, and an electrode having recording and stimulating capabilities.

69. The method of claim 66, further comprising detecting electrical signals with the multi-electrode array, the electrical signals being indicative of a neural activity preceding the neurological event.

70. The method of claim 66, further comprising detecting electrical signals generated from the neurons located proximate the implant.

71. The method of claim 70, further comprising processing the detected electrical signals to convert the signals into a recognizable pattern.

72. The method of claim 71, wherein the recognizable pattern includes a formula describing a behavior of the neurons in time and space.

73. The method of claim 66, further comprising processing the detected electrical signals to isolate individual neuron signals from neighboring neuron signals.

74. The method of claim 66, wherein the detected electrical signals generated from the neurons include electrical spikes.

75. The method of claim 65, wherein the implant is placed proximate a neural focus in the brain that initiates the neurological event.

76. The method of claim 65, wherein the implant is configured detect local field potentials of the brain.

77. The method of claim 65, wherein the implant is configured to detect electrocorticogram (ECoG) signals.

78. The method of claim 65, wherein the implant is configured to detect electroencephalogram (EEG) signals.

79. The method of claim 65, wherein the implant is configured to detect DC potentials.

80. The method of claim 65, wherein the implant is configured to detect light generated from the neurons located proximate the implant.

81. The method of claim 65, wherein the implant is configured to detect acoustic waves generated from the neurons located proximate the implant.

82. The method of claim 65, wherein the implant comprises a subdural grid having a plurality of electrode contacts, the subdural grid being placed on a surface of the brain.

83. The method of claim 82, wherein the implant further comprises at least one multi-electrode array.

84. The method of claim 65, wherein the implant includes a movement sensor configured to detect movement of the brain.

85. The method of claim 65, wherein the implant includes a pressure monitoring device for monitoring pressure in the brain.

86. The method of claim 65, wherein the implant includes a temperature monitoring device for monitoring temperature in the brain.

87. The method of claim 65, wherein the implant includes a magnetic resonance monitoring device for monitoring magnetic resonance intensity in the brain.

88. The method of claim 64, further comprising processing the detected signals to characterize the signals that represent the activity preceding the neurological event.

89. The method of claim 64, further comprising storing the signals that represent the activity preceding the neurological event into a storage device.

90. The method of claim 89, further comprising comparing the detected signals with the signals stored in the storage device.

91. The method of claim 64, further comprising comparing the detected signals with other signals detected by a sensor in the patient's body.

92. The method of claim 64, further comprising recording the detected signals.

93. The method of claim 64, wherein the detected signals include biological or physiological signals generated within the patient's body.

94. The method of claim 64, further comprising differentiating the signals indicative of the activity that precedes the neurological event from signals resulting from normal activities.

95. The method of claim 64, further comprising outputting information relating to the patient's condition with respect to the neurological event.

96. The method of claim 95, wherein outputting information includes conveying the information to the patient.

97. The method of claim 95, wherein the information includes a warning signal that the neurological event is expected to occur.

98. The method of claim 95, wherein outputting information includes communicating with an external device to convey the information.

99. The method of claim 1 16, wherein the external device includes at least one of a visual indicator, a tactile transducer, and an auditory indicator.

100. The method of claim 95, wherein the information includes a time remaining until the occurrence of the neurological event.

101. The method of claim 95, wherein the information includes an occurrence probability of the neurological event.

102. The method of claim 95, wherein the information includes severity of the neurological event.

103. The method of claim 95, wherein the information includes a patient's current condition in comparison with a normal target condition.

104. The method of claim 95, wherein the information includes instructions for preventing the neurological event from occurring.

105. The method of claim 95, wherein outputting the information includes causing a movement of a portion of the patient's body.

106. The method of claim 105, wherein causing the movement includes sending a stimulating signal to the implant.

107. The method of claim 105, wherein the portion of the patient's body includes a finger.

108. The method of claim 64, further comprising, upon predicting the occurrence of the neurological event, generating a control signal for treating the patient.

109. The method of claim 108, wherein the control signal controls, suppresses, dampens, and/or delays the neurological event.

110. The method of claim 108, wherein generating a control signal includes generating a stimulating electrical current and sending the current to a portion of the patient's body.

111. The method of claim 110, wherein the portion of the patient's body includes the patient's brain.

112. The method of claim 108, wherein generating a control signal includes generating a signal to deliver a drug or a therapeutic agent to at least a portion of the patient's body.

113. The method of claim 64, further comprising, upon predicting the occurrence of the neurological event, hyperpolarizing at least a portion of the patient's brain.

114. The method of claim 113, wherein hyperpolarizing includes sending a DC bias current to the patient's brain to hyperpolarize the at least a portion of the brain.

115. The method of claim 64, wherein:
the implant includes at least one electrode; and
upon predicting the occurrence of the neurological event, the processing unit is configured to short the at least one electrode.

116. The method of claim 64, further comprising providing a target signal indicative of the activity that precedes the neurological event.

117. The method of claim 116, wherein the target signal includes a database containing a set of previously detected signals indicative of the activity that precedes the neurological event.

118. The method of claim 116, further comprising comparing the detected signals with the target signal.

119. The method of claim 116, further comprising modifying the target signal.

120. The method of claim 119, wherein modifying the target signal includes performing an adaptive processing of the target signal.

121. The method of claim 119, wherein the adaptive processing includes:
determining whether the neurological event occurred, regardless of whether the occurrence was predicted;
determining whether the occurrence or nonoccurrence of the neurological event was mistakenly predicted; and
modifying the target signal based on whether the occurrence or nonoccurrence of the neurological event was mistakenly predicted.

122. The method of claim 64, wherein the neurological event is an epileptic symptom.

123. The method of claim 122, further comprising placing the implant proximate an epileptic focus of a brain.

124. The method of claim 64, further comprising preprocessing the detected signal.

125. The method of claim 124, wherein preprocessing includes measuring background signals and calibrating the detected signal based on the measured background signals.

126. The method of claim 124, wherein preprocessing includes at least one of: noise filtering, impedance matching, rectifying, integrating, differentiating, discretizing, and amplifying the detected signals.

127. A system for detecting a neurological event in a patient's body, comprising:
at least one electrode placed within a patient's brain and configured to detect electrical signals generated from the brain; and
a control module in communication with the at least one electrode and comprising:
an event detection device configured to identify occurrence of the neurological event based on the detected electrical signals; and
a data recording device including a counter synchronized with an external clock;
wherein, upon identifying occurrence of the neurological event by the event detection device, the recording device is configured to record the detected electrical signals and a value of the counter.

128. The system of claim 127, wherein the value of the counter is configured to increase by one in every predetermined time interval.

129. The system of claim 127, further comprising an external device configured to communicate with the control module, wherein the external device is configured to receive the value of the counter and the detected electrical signals from the remote module.

130. The system of claim 129, wherein the external device is configured to convert the value of the counter to a real-time value.

131. The system of claim 129, wherein the external device is configured to transmit a start signal to the control module to upload the value of the counter and the detected electrical signals to the external device or other processing device.

132. The system of claim 129, wherein the external device is configured to receive a start signal to the remote module or other processing device to download the value of the counter and the detected electrical signals from the control module.

133. The system of claim 127, wherein the at least one electrode includes at least one multi-electrode array, the multi-electrode array including a plurality of electrodes.

134. The system of claim 133, wherein the plurality of electrodes are configured to penetrate into neural tissue of the brain to detect electrical signals generated from the neurons.

135. The system of claim 127, wherein the at least one electrode is configured to detect local field potentials of the brain.

136. The system of claim 127, wherein the at least one electrode is configured to detect electrocorticogram (ECoG) signals.

137. The system of claim 127, wherein the at least one electrode is configured to detect electroencephalogram (EEG) signals.

138. A device for placing an implant in a patient's body, comprising:
an elongated member having a distal sleeve, the distal sleeve having a first portion and a second portion and configured to receive the implant between the first portion and the second portion, the first portion and the second portion being configured to move relative to each other,
wherein at least the first portion includes an expandable member so as to push the implant towards an implant site in the patient's body.

139. The device of claim 138, wherein the elongated member is configured to be bent or turned.

140. The device of claim 138, wherein the elongated member is sufficiently flexible to traverse through tortuous paths within the patient's body.

141. The device of claim 138, further comprising the implant, wherein the implant includes a plurality of electrodes for placement in a brain of the patient, and wherein at least one of the first portion and the second portion is configured to cover the plurality of electrodes.

142. The device of claim 138, wherein the first portion is inflatable.

143. The device of claim 138, further comprises a substantially rigid backing member, wherein the first portion is configured to push against the backing member to expand towards an implant site.

144. The device of claim 138, further comprising a grasping member to grasp the implant.

145. The device of claim 138, wherein at least a portion of the device is made of a bioabsorbable material.

146. A system for detecting occurrence of an undesired activity in a person, comprising:
an implant configured to be placed in the body and detect signals indicating that the undesired activity is occurring or is about to occur; and
a processing unit configured to process the detected signals and generate a control signal to prevent the undesired activity and/or warn the person or a third person.

147. The system of claim 146, wherein the control signal is at least one of an electrical signal and a chemical signal.

148. The system of claim 146, wherein the control signal is inputted to the brain.

149. The system of claim 146, wherein the control signal is inputted to at least a portion of the central nervous system and/or peripheral nervous system to prevent the undesired activity.

150. A system for detecting and treating a neurological event in a patient's body, comprising:
an implant configured to be placed in the body and detect signals generated from the body;
an external device; and
a processing unit configured to process the detected signals and generate a control signal that controls the operation of the external device.

151. The system of claim 151, wherein the external device is a movement device, the movement of the device being controlled by the processing unit.
